(19) United States
(12) Patent Application Publication
(10) Pub. No.: US 2015/0051677 A1
(43) Pub. Date: Feb. 19, 2015

(54) CONTROL MODULE WITH PORT FOR RECEIVING ONE OR TWO LEADS AND SYSTEMS AND METHODS USING THE CONTROL MODULE

(71) Applicant: BOSTON SCIENTIFIC NEUROMODULATION CORPORATION, Valencia, CA (US)

(72) Inventor: Goran N. Marnfeldt, Valencia, CA (US)

(21) Appl. No.: 14/457,703

(22) Filed: Aug. 12, 2014

Related U.S. Application Data
(60) Provisional application No. 61/867,428, filed on Aug. 19, 2013.

(51) Int. Cl. A61N 1/375

(52) U.S. Cl. CPC A61N 1/3752
USPC 607/116

(57) ABSTRACT

One embodiment is an implantable control module for coupling to one or more implantable stimulation leads. The control module includes a sealed housing, an electronic subassembly disposed in the housing, a header arrangement coupled to the housing, and a number of feedthrough elements. The header arrangement includes at least one receiving lumen and a number of contacts disposed within the at least one lumen. Each receiving lumen has two opposing openings which can receive an implantable stimulation lead through the opening and within the receiving lumen. The contacts are arranged within the at least one receiving lumen to make contact with (or otherwise be in electrical communication with) terminals at or on the stimulation lead received in the receiving lumen. The feedthrough elements extend from the header arrangement into the sealed housing, and electrically couple the contacts of the header arrangement with the electronic subassembly.

### CROSS-REFERENCE TO RELATED APPLICATIONS

This application claims the benefit under 35 U.S.C. §119(e) of U.S. Provisional Patent Application Ser. No. 61/867,428, filed Aug. 19, 2013, which is incorporated herein by reference.

### FIELD

The present invention is directed to the area of implantable electrical stimulation systems and methods of making and using the systems. The present invention is also directed to control modules having a port for receiving one or two implantable electrical stimulation leads, as well as methods of making and using the control modules and electrical stimulation systems.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

### BRIEF SUMMARY

One embodiment is an implantable control module for coupling to one or more implantable stimulation leads. The control module includes a sealed housing, an electronic subassembly disposed in the housing, a header arrangement coupled to the housing, and a number of feedthrough elements. The header arrangement includes at least one receiving lumen and a number of contacts disposed within the at least one lumen. Each receiving lumen has two opposing openings, each of which can receive an implantable stimulation lead through the opening and within the receiving lumen. The contacts are arranged within the at least one receiving lumen to make contact with (or otherwise be in electrical communication with) terminals at or on the implantable stimulation lead received in the receiving lumen. The feedthrough elements can extend from the header arrangement into the sealed housing, and can electrically couple the contacts of the header arrangement with the electronic subassembly.

Another embodiment is a kit including the control module as described above and at least one lead. The lead includes a proximal end, which can be inserted into one of the at least one receiving lumen of the control module.

Yet another embodiment includes a method of implanting an electrical stimulation lead. The method includes providing a control module as described above. The method also includes inserting a proximal end portion of a first electrical stimulation lead into a first opening of a first receiving lumen of the at least one receiving lumen of the control module. The method further includes implanting the first electrical stimulation lead and the control module.

Still other embodiments are directed to manufacturing the apparatus disclosed above, such as the control module, kit, etc.

### BRIEF DESCRIPTION OF THE DRAWINGS

Non-limiting and non-exhaustive embodiments of the present invention are described with reference to the following drawings. In the drawings, like reference numerals refer to like parts throughout the various FIGS. unless otherwise specified.

For a better understanding of the present invention, reference will be made to the following Detailed Description, which is to be read in association with the accompanying drawings, wherein:

FIG. 1 is a schematic view of one embodiment of an electrical stimulation system that includes a paddle lead electrically coupled to a control module;

FIG. 2 is a schematic view of one embodiment of an electrical stimulation system that includes a percutaneous lead electrically coupled to a control module;

FIG. 3A is a schematic view of one embodiment of the control module of FIG. 1 configured and arranged to electrically couple to an elongated device;

FIG. 3B is a schematic view of one embodiment of a lead extension configured and arranged to electrically couple the elongated device of FIG. 2 to the control module of FIG. 1;

FIG. 4A is a schematic side view of one embodiment of a control module, according to the invention;

FIG. 4B is a schematic side view of one embodiment of an arrangement of a lead and plug for insertion in the receiving lumen of the control module of FIG. 4A, according to the invention;

FIG. 4C is a schematic side view of one embodiment of an arrangement of two leads for insertion in the receiving lumen of the control module of FIG. 4A, according to the invention;

FIG. 4D is a schematic side view of one embodiment of an arrangement of a lead for insertion in the receiving lumen of the control module of FIG. 4A, according to the invention;

FIG. 4E is a schematic partial-cross-sectional top view of the header arrangement of the control module of FIG. 4A, according to the invention;

FIG. 5A is a schematic side view of another embodiment of a control module, according to the invention;

FIG. 5B is a schematic side view of one embodiment of an arrangement of a lead and plug for insertion in the receiving lumen of the control module of FIG. 5A, according to the invention;

FIG. 5C is a schematic side view of one embodiment of an arrangement of two leads for insertion in the receiving lumen of the control module of FIG. 5A, according to the invention;

FIG. 5D is a schematic side view of one embodiment of an arrangement of another lead and a plug for insertion in the receiving lumen of the control module of FIG. 5A, according to the invention;

FIG. 5E is a schematic partial-cross-sectional top view of the header arrangement of the control module of FIG. 5A, according to the invention; and

FIG. 6 is a schematic overview of one embodiment of components of a stimulation system, including an electronic subassembly disposed within a control module, according to the invention.

### DETAILED DESCRIPTION

The present invention is directed to the area of implantable electrical stimulation systems and methods of making and using the systems. The present invention is also directed to control modules having a port for receiving one or two implantable electrical stimulation leads, as well as methods of making and using the control modules and electrical stimulation systems.

Suitable implantable electrical stimulation systems include, but are not limited to, a least one lead with one or more electrodes disposed along a distal end of the lead and one or more terminals disposed along the one or more proximal ends of the lead. Leads include, for example, percutaneous leads, paddle leads, and cuff leads. Examples of electrical stimulation systems with leads are found in, for example, U.S. Pat. Nos. 6,181,969; 6,516,227; 6,609,029; 6,609,032; 6,741,892; 7,949,395; 7,244,150; 7,672,734; 7,761,165; 7,974,706; 8,175,710; 8,224,450; and 8,364,278; and U.S. Patent Application Publication No. 2007/0150036, all of which are incorporated by reference.

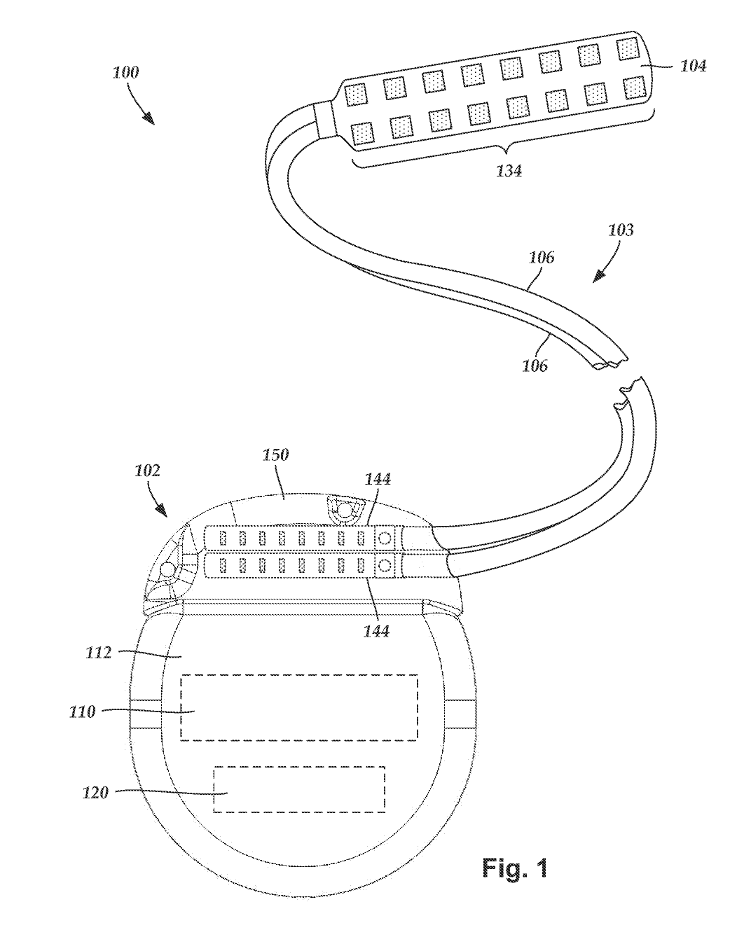

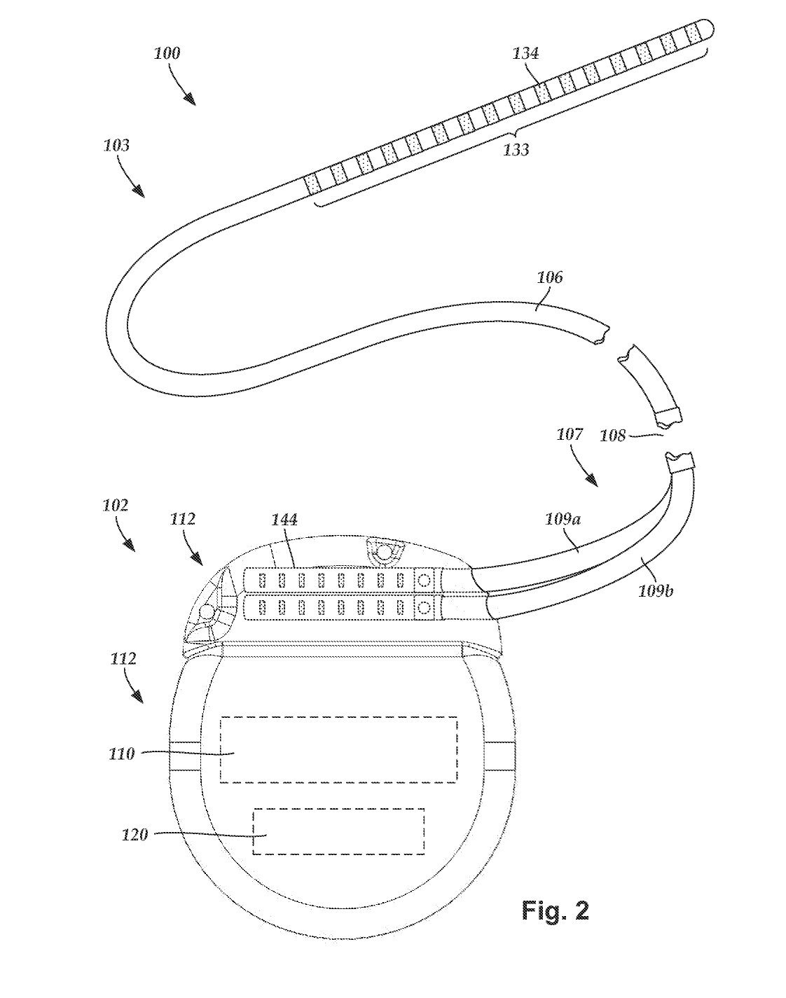

FIG. 1 illustrates schematically one embodiment of an electrical stimulation system 100. The electrical stimulation system includes a control module (e.g., a stimulator or pulse generator) 102 and a lead 103 coupleable to the control module 102. The lead 103 includes a paddle body 104 and one or more lead bodies 106. In FIG. 1, the lead 103 is shown having two lead bodies 106. It will be understood that the lead 103 can include any suitable number of lead bodies including, for example, one, two, three, four, five, six, seven, eight or more lead bodies 106. An array of electrodes 133, such as electrode 134, is disposed on the paddle body 104, and an array of terminals (e.g., 210 in FIG. 2A-2B) is disposed along each of the one or more lead bodies 106.

It will be understood that the electrical stimulation system can include more, fewer, or different components and can have a variety of different configurations including those configurations disclosed in the electrical stimulation system references cited herein. For example, instead of a paddle body, the electrodes can be disposed in an array at or near the distal end of a lead body forming a percutaneous lead.

FIG. 2 illustrates schematically another embodiment of the electrical stimulation system 100, where the lead 103 is a percutaneous lead. In FIG. 2, the electrodes 134 are shown disposed along the one or more lead bodies 106. In at least some embodiments, the lead 103 is isodiametric along a longitudinal length of the lead body 106.

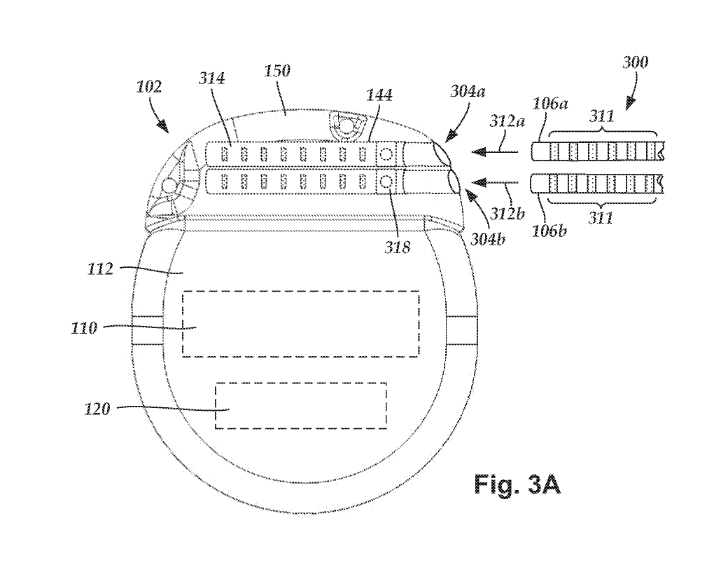

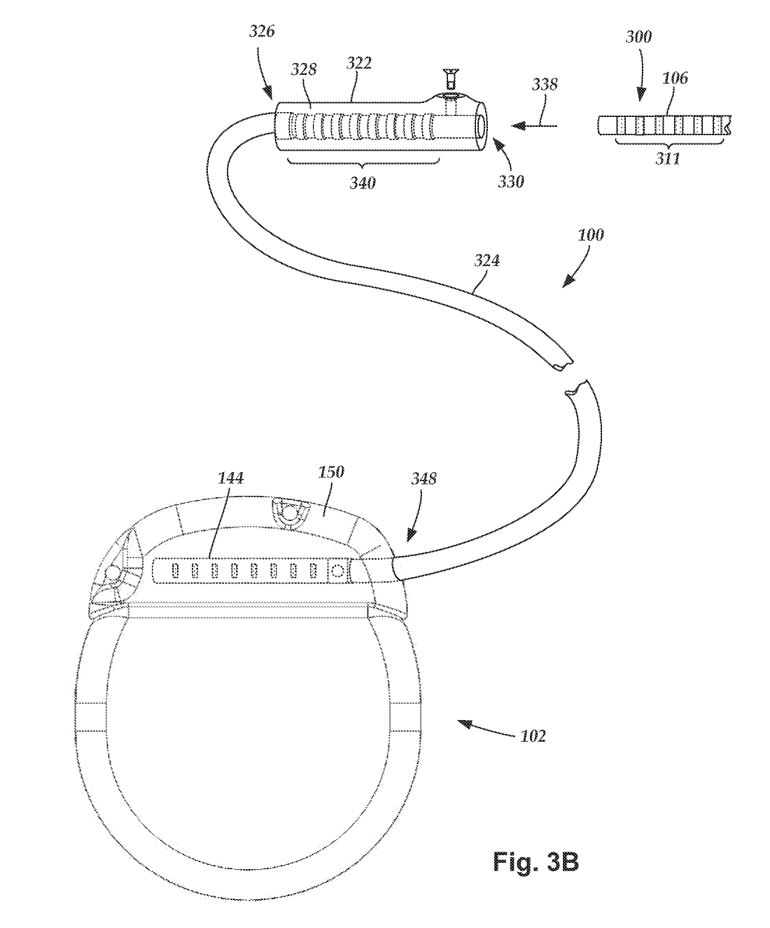

The lead 103 can be coupled to the control module 102 in any suitable manner. In FIG. 1, the lead 103 is shown coupling directly to the control module 102. In at least some other embodiments, the lead 103 couples to the control module 102 via one or more intermediate devices (300 in FIGS. 3A-3B). For example, in at least some embodiments one or more lead extensions 324 (see e.g., FIG. 3B) can be disposed between the lead 103 and the control module 102 to extend the distance between the lead 103 and the control module 102. Other intermediate devices may be used in addition to, or in lieu of, one or more lead extensions including, for example, a splitter, an adaptor, or the like or combinations thereof. It will be understood that, in the case where the electrical stimulation system 100 includes multiple elongated devices disposed between the lead 103 and the control module 102, the intermediate devices may be configured into any suitable arrangement.

In FIG. 2, the electrical stimulation system 100 is shown having a splitter 207 configured and arranged for facilitating coupling of the lead 103 to the control module 102. The splitter 207 includes a splitter connector 208 configured to couple to a proximal end of the lead 103, and one or more splitter tails 209a and 209b configured and arranged to couple to the control module 102 (or another splitter, a lead extension, an adaptor, or the like).

The control module 102 typically includes a header arrangement 150 and a sealed electronics housing 112. An electronic subassembly 110 and an optional power source 120 are disposed in the electronics housing 112. A control module connector 144 is disposed in the header arrangement 150. The control module connector 144 is configured and arranged to make an electrical connection between the lead 103 and the electronic subassembly 110 of the control module 102.

The electrical stimulation system or components of the electrical stimulation system, including the paddle body 104, the one or more of the lead bodies 106, and the control module 102, are typically implanted into the body of a patient. The electrical stimulation system can be used for a variety of applications including, but not limited to deep brain stimulation, neural stimulation, spinal cord stimulation, muscle stimulation, and the like.

The electrodes 134 can be formed using any conductive, biocompatible material. Examples of suitable materials include metals, alloys, conductive polymers, conductive carbon, and the like, as well as combinations thereof. In at least some embodiments, one or more of the electrodes 134 are formed from one or more of: platinum, platinum iridium, palladium, palladium rhodium, or titanium.

Any suitable number of electrodes 134 can be disposed on the lead including, for example, four, five, six, seven, eight, nine, ten, eleven, twelve, fourteen, sixteen, twenty-four, thirty-two, or more electrodes 134. In the case of paddle leads, the electrodes 134 can be disposed on the paddle body 104 in any suitable arrangement. In FIG. 1, the electrodes 134 are arranged into two columns, where each column has eight electrodes 134.

The electrodes of the paddle body 104 (or one or more lead bodies 106) are typically disposed in, or separated by, a non-conductive, biocompatible material such as, for example, silicone, polyurethane, polyetheretherketone (“PEEK”), epoxy, and the like or combinations thereof. The one or more lead bodies 106 and, if applicable, the paddle body 104 may be formed in the desired shape by any process including, for example, molding (including injection molding), casting, and the like. The non-conductive material typically extends from the distal ends of the one or more lead bodies 106 to the proximal end of each of the one or more lead bodies 106.

In the case of paddle leads, the non-conductive material typically extends from the paddle body 104 to the proximal end of each of the one or more lead bodies 106. Additionally, the non-conductive, biocompatible material of the paddle body 104 and the one or more lead bodies 106 may be the same or different. Moreover, the paddle body 104 and the one or more lead bodies 106 may be a unitary structure or can be formed as two separate structures that are permanently or detachably coupled together.

Terminals (e.g., 311 in FIGS. 3A-3B) are typically disposed along the proximal end of the one or more lead bodies 106 of the electrical stimulation system 100 (as well as any splitters, lead extensions, adaptors, or the like) for electrical connection to corresponding connector contacts (e.g., 314 in FIGS. 3A-3B). The connector contacts are disposed in connectors (e.g., 144 in FIGS. 1-3B; and 322 FIG. 3B) which, in turn, are disposed on, for example, the control module 102 (or a lead extension, a splitter, an adaptor, or the like). Electrically conductive wires, cables, or the like (not shown) extend from the terminals to the electrodes 134. Typically, one or more electrodes 134 are electrically coupled to each terminal. In at least some embodiments, each terminal is only connected to one electrode 134.

The electrically conductive wires (“conductors”) may be embedded in the non-conductive material of the lead body 106 or can be disposed in one or more lumens (not shown) extending along the lead body 106. In some embodiments, there is an individual lumen for each conductor. In other embodiments, two or more conductors extend through a lumen. There may also be one or more lumens (not shown) that open at, or near, the proximal end of the one or more lead bodies 106, for example, for inserting a stylet to facilitate placement of the one or more lead bodies 106 within a body of a patient. Additionally, there may be one or more lumens (not shown) that open at, or near, the distal end of the one or more lead bodies 106, for example, for infusion of drugs or medication into the site of implantation of the one or more lead bodies 106. In at least one embodiment, the one or more lumens are flushed continually, or on a regular basis, with saline, epidural fluid, or the like. In at least some embodiments, the one or more lumens are permanently or removably sealable at the distal end.

FIG. 3A is a schematic side view of one embodiment of a proximal end of one or more elongated devices 300 configured and arranged for coupling to one embodiment of the control module connector 144. The one or more elongated devices may include, for example, one or more of the lead bodies 106 of FIG. 1, one or more intermediate devices (e.g., a splitter, the lead extension 324 of FIG. 3B, an adaptor, or the like or combinations thereof), or a combination thereof.

The control module connector 144 defines at least one port into which a proximal end of the elongated device 300 can be inserted, as shown by directional arrows 312a and 312b. In FIG. 3A (and in other FIGS.), the header arrangement 150 is shown having two ports 304a and 304b. The header arrangement 150 can define any suitable number of ports including, for example, one, two, three, four, five, six, seven, eight, or more ports.

The control module connector 144 also includes a plurality of connector contacts, such as connector contact 314, disposed within each port 304a and 304b of a header arrangement 150. When the elongated device 300 is inserted into the ports 304a and 304b, the connector contacts 314 can be aligned with a plurality of terminals 311 disposed along the proximal end(s) of the elongated device(s) 300 to electrically couple the control module 102 to the electrodes (134 of FIG. 1) disposed on the paddle body 104 of the lead 103. Examples of connectors in control modules are found in, for example, U.S. Pat. Nos. 7,244,150 and 8,224,450, which are incorporated by reference. The control module 102 can also include a retention assembly 318 that can include a fastener within a retention lumen that can be tightened onto the lead (or a retention sleeve of a lead) to hold the lead within the port.

FIG. 3B is a schematic side view of another embodiment of the electrical stimulation system 100. The electrical stimulation system 100 includes a lead extension 324 that is configured and arranged to couple one or more elongated devices 300 (e.g., one of the lead bodies 106 of FIGS. 1 and 2, the splitter 207 of FIG. 2, an adaptor, another lead extension, or the like or combinations thereof) to the control module 102. In FIG. 3B, the lead extension 324 is shown coupled to a single port 304 defined in the control module connector 144. Additionally, the lead extension 324 is shown configured and arranged to couple to a single elongated device 300. In alternate embodiments, the lead extension 324 is configured and arranged to couple to multiple ports 304 defined in the control module connector 144, or to receive multiple elongated devices 300, or both.

A lead extension connector 322 is disposed on the lead extension 324. In FIG. 3B, the lead extension connector 322 is shown disposed at a distal end 326 of the lead extension 324. The lead extension connector 322 includes a connector housing 328. The connector housing 328 defines at least one port 330 into which terminals 311 of the elongated device 300 can be inserted, as shown by directional arrow 338. The connector housing 328 also includes a plurality of connector contacts, such as connector contact 340. When the elongated device 300 is inserted into the port 330, the connector contacts 240 disposed in the connector housing 328 can be aligned with the terminals 311 of the elongated device 300 to electrically couple the lead extension 324 to the electrodes (134 of FIGS. 1 and 2) disposed along the lead (103 in FIGS. 1 and 2).

In at least some embodiments, the proximal end of the lead extension 324 is similarly configured and arranged as a proximal end of the lead 103 (or other elongated device 300). The lead extension 324 may include a plurality of electrically conductive wires (not shown) that electrically couple the connector contacts 340 to a proximal end 348 of the lead extension 324 that is opposite to the distal end 326. In at least some embodiments, the conductive wires disposed in the lead extension 324 can be electrically coupled to a plurality of terminals (not shown) disposed along the proximal end 348 of the lead extension 324. In at least some embodiments, the proximal end 348 of the lead extension 324 is configured and arranged for insertion into a connector disposed in another lead extension (or another intermediate device). In other embodiments (and as shown in FIG. 3B), the proximal end 348 of the lead extension 324 is configured and arranged for insertion into the control module connector 144.

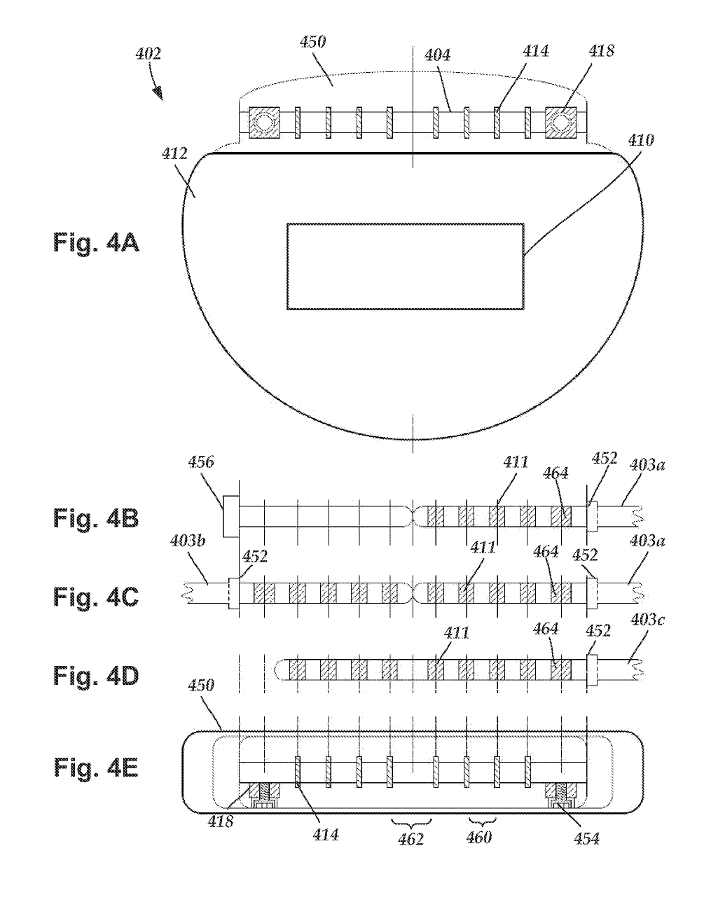

FIG. 4A is a schematic side view of one embodiment of a control module 402. The control module 402 can be implanted within a patient's body. The control module 402 includes a housing 412, and an electronic subassembly 410 disposed within the housing 412. The housing 412 may be a sealed housing 412, the sealing of the housing 412 can resist or prevent moisture or fluid penetration into the housing 412, and in turn may resist or prevent potential damage to internal components of the housing 412, such as, the electronic subassembly 410. The housing 412 may be formed using any biocompatible material suitable to protect the components located within the housing 412. Further, the electronic subassembly 410 may include one or more components electrically coupled to each other, such that the electronic subassembly can generate electronic pulses that may be delivered to the patient via one or more stimulation leads.

The control module 402 can couple to one or more implantable stimulation leads (such as, a lead 403a, a lead 403b, or a lead 403c as shown in FIGS. 4B-4D). Further, as discussed with reference to FIGS. 4B-4D, the lead 403a-403b can be a short lead having at least four terminals 411 (i.e., 1×4 lead), or the lead 403c can be a long lead having at least eight terminals 411 (i.e., 1×8 lead). The number of terminals on the illustrated leads is simply provided for an example. It will be understood that leads with different numbers of terminals and corresponding control modules with sufficient conductive contacts can be used. Each of the leads 403a-403c can include a lead body (not shown) having one or more electrodes disposed at a distal end of the lead 403a-403c and multiple terminals 411 disposed at a proximal end of the lead 403a-403c. In some embodiments, the control module 402 may deliver electrical stimulation or current to one or more electrodes.

The control module 402 can provide stimulation signals, such as electrical pulses, to the electrodes of the leads 403a-403c (for ultimate delivery to body tissue) via a header arrangement 450 coupled to the housing 412. The header arrangement 450 can be formed using any suitable non-conductive, biocompatible material such as, silicone, polyurethane, polyetheretherketone (“PEEK”), epoxy, PVC, and the like, or combinations thereof. Further, the header arrangement 450 can be coupled to the housing 412 through various suitable techniques, such as, but not limited to, adhesive bonding, heat bonding, and so forth. The header arrangement 450 includes at least one receiving lumen 404 and a number of contacts 414 disposed around the circumference of the receiving lumen 404.

In some embodiments, the contacts 414 may be spaced apart uniformly within the receiving lumen 404, such that there is a substantial distance between two adjacent contacts 414. In the embodiment shown in FIG. 4A, all of the contacts 414 are spaced from each other by the same or substantially the same predefined distance. In some embodiments, the contacts of the middle pair of contacts 414 are spaced at twice or substantially twice the predefined distance. The contacts are spaced twice so as to accommodate tip portions of the two short leads 403a-403b, when the two leads are disposed within the receiving lumen 404.

Embodiments are intended to cover any number of lumens, including one or more than one receiving lumen 404. The receiving lumen 404 can be considered as a bore within the header arrangement 450 to receive one or more of the leads 403a-403c. The receiving lumen 404 can be formed into the header arrangement 450 using any suitable manufacturing technique, such as, but not limited to, machining, drilling, ablation, molding, and so forth. Further, the receiving lumen 404 can have various suitable cross-sections that are at least operationally consistent with the cross-section of the lead (leads 403a-403c). In other words, the receiving lumen 404 can have any shape or cross-section that enables entry of the lead(s) and electrical communication between the terminals 411 and the contacts 414. Examples of the suitable cross-sections include, but are not limited to, elliptical, circular, oval, and so forth. Further, the receiving lumen 404 may have an unsymmetrical shape and structure that are operationally consistent with shape, size and structure of the lead in ways similar to those discussed above.

The receiving lumen 404 also includes at least two openings, such as a first opening and a second opening, at two opposite ends (i.e., a first end and a second end) of the receiving lumen 404. Each of the openings can receive one lead and two leads can be disposed in the receiving lumen 404 simultaneously via the two openings. The contacts 414 are arranged within the receiving lumen 404, so as to make contact with the terminals 411 of the one or more leads 403a-403c. The terminals 411 can be disposed in, or separated by, a non-conductive, biocompatible material, such as silicone.

The control module 402 also includes a number of feedthrough elements extending from the header arrangement 450 into the housing 412. The feedthrough elements electrically couple the contacts 414 of the header arrangement 450 with the electronic subassembly 410. In some embodiments, the feedthrough elements can include a pin made of a conducting material, such as, metal, alloy, or the like. Further, the feedthrough elements may be formed separately and can be attached to the contacts using any suitable technique, such as, but not limited to, soldering, welding, and the like. In some embodiments, the feedthrough elements may form a unitary structure with the contacts 414.

Insertion of one or more leads into the receiving lumen will be described with reference to FIGS. 4B-4D. FIG. 4B shows a schematic side view of one embodiment of an arrangement of a single lead 403a and a plug 456 for insertion in the receiving lumen 404 of the control module 402. In particular, as illustrated in FIG. 4B, one short lead 403a can be inserted into the first opening of the receiving lumen 404 and the long plug 456 can be inserted into the second opening of the receiving lumen 404. The long plug 456 may seal the receiving lumen 404 from one of the ends and may impede or prevent entry of fluid, such as blood, or any other foreign particle into the receiving lumen 404 when the control module 402 is implanted. In at least some embodiments, the long plug 456 is dimensioned such that when fully inserted within the receiving lumen 404, the long plug 456 extends past at least half of the contacts 414 in the receiving lumen 404. As shown, a distal end of the long plug 456 may contact (or nearly contact) the end portion of the lead 403a. The long plug 456 may be formed using any suitable biocompatible material, such as, but not limited to, PVC, plastic, silicone, or the like. When the plug 456 is inserted in one of the openings, then only one short lead, i.e., lead 403a, can be disposed within the receiving lumen 404.

In the illustrated embodiment, the lead 403a includes four lead terminals 411. Any suitable number of the terminals 411 can be disposed on the lead 403a including, for example, four, five, six, seven, eight, nine, ten, eleven, twelve, fourteen, sixteen, twenty-four, thirty-two, or more terminals.

The lead 403a also includes a flange 452, which can be a solid body disposed over the lead 403a. In some embodiments, the flange 452 has dimensions greater than dimensions of the opening(s) of the receiving lumen 404, so that the flange 452 will remain outside the receiving lumen 404. The flange 452 can facilitate or ensure that the lead 403a is inserted within the receiving lumen 404 up to (or only to) a particular predefined length or level. When the lead 403a is disposed within the receiving lumen 404 through one of the openings, the flange 452 sits or is disposed outside of the opening, such that, the lead terminals 411 are aligned with the contacts 414 of the receiving lumen 404. The flange 452 may form a unitary structure with the lead 403a. Further, the flange 452 can be created using any suitable technique, such as, but not limited to, casting, molding, machining, and so forth. In some embodiments, the flange 452 may be integrated over (or otherwise be a separate element that is attached to) the lead 403a. Further, the flange 452 can be manufactured using the same material as that of the lead 403a.

The lead 403a also includes a retention sleeve 464 disposed proximal to a most proximal terminal 411. The retention sleeve 464 may be or become aligned with the retention assembly 418 in order to protect the lead 403a from damage from the retention assembly 418.

The receiving lumen 404 can accommodate two short leads. FIG. 4C shows a schematic side view of one embodiment of an arrangement of the two leads 403a, 403b, for insertion in the receiving lumen 404 of the control module 402. The two short leads 403a, 403b can be disposed within the receiving lumen 404 from the first opening and second opening of the receiving lumen 404, respectively. As shown, the leads 403a and 403b are structurally similar, but it will be understood that structurally different leads could also be used so long as the terminals of the leads can be aligned with the contacts of the receiving lumen. The leads 403a, 403b can be disposed in the receiving lumen 404 such that the flanges 452 of both of the leads 403a, 403b sit or are disposed outside the respective openings. When the flange 452 makes contact with the header arrangement 450, further insertion of the lead can be halted or stopped. The flange 452 may also ensure proper insertion of the leads 403a, 403b in the receiving lumen and proper alignment of the terminals 411 with the contacts 414. If leads 403a, 403b are fully disposed in the receiving lumen 404, then both the leads 403a-403b can extend past half the contacts 414 in the receiving lumen 404. The lead terminals 411 of the leads 403a, 403b, respectively, may be or become aligned with the respective contacts 414 in the receiving lumen 404. Further, the middle pair of contacts 414 may be spaced to accommodate the tip portions of the leads 403a-403 properly. For example, in an embodiment, the middle pair of contacts is spaced at a distance which is twice to the size of a tip portion of the lead 403a or equal to the sum of the size of the tip portions of the two leads 403a, 403b.

FIG. 4D is a schematic side view of one embodiment of an arrangement of a long lead 403c for insertion in the receiving lumen 404 of the control module 402. The long lead 403c includes terminals 411, which are more than half the number of contacts 414 in the header arrangement 450. As shown, the long lead 403c includes eight lead terminals 411 which is equal to the number of contacts 414 in the receiving lumen 404. The lead terminals 411 are arranged in a similar manner on the lead 403c as they are arranged on the lead 403a. This arrangement may enable insertion of two small leads 403a, 403b simultaneously in the receiving lumen 404 from two openings at two ends of the receiving lumen 404. The increased (double) distance between the middle pair of the contacts 414 can accommodate the respective tip portions of the leads 403a, 403b. Further, when disposed within the receiving lumen 404, a middle pair of terminals 411 of the lead 403c is spaced at a distance greater than that of a distance between other pairs of lead terminals 411 in order to align with the contacts 414.

The lead 403c also includes a flange similar in structure and function to the flange 452. The flange 452 allows the lead 403c to enter into the receiving lumen 404 up to a certain or defined extent. Further, the flange 452 may ensure that the terminals 411 are properly aligned with the contacts 414.

FIG. 4E is a schematic partial-cross-sectional top view of the header arrangement 450 of the control module 402. The contacts 414 are spaced apart from each other, such that there is a substantial distance between the two adjacent contacts 414. In some embodiments, the contacts 414 are uniformly spaced apart, such that each adjacent pair of contacts 414 is spaced apart by a first distance. Further, a middle pair of the contacts 414 is spaced apart by a second distance, and the first distance is different than the second distance. In some embodiments, the second distance may be double the first distance.

The header arrangement 450 also includes a retention assembly 418 that defines a retention lumen (not shown) for holding a fastener 454. The retention lumen may further include threads, groves, channel, ridges or the like to engage the fastener 454. The fastener 454 may include threads, ridges, and so forth corresponding to the threads, groves, channel, ridges or the like of the retention lumen. The fastener 454 can be tightened onto the lead (lead 403a-403c) to secure the lead within the receiving lumen 404, such as by pressure or contact. In some embodiments, the retention assembly 418 may include any mechanism other than the fastener 454 to secure the lead within the receiving lumen 404.

As one example, a physician may couple the control module 402 with the lead or leads (e.g., lead 403a) before or after implanting in a patient. A proximal end portion of the lead 403a may be inserted into the receiving lumen 404 of the control module 402 through either of the first and the second openings of the receiving lumen 404. The short lead 403a, when disposed, may extend past only half of the contacts in the receiving lumen 404. In another example, where the patient is to be implanted with more than one lead, the physician inserts one lead 403a into the first opening and the second lead 403b into the second opening. In another example, when only one short lead 403a is inserted into the first opening, the physician may insert the plug 456, such as the long plug 456, into the second opening of the receiving lumen 404 to block the opening. The plug 456 may disposed into the receiving lumen 404 such that the plug 456 can extend past at least half of the contacts 414 within the receiving lumen 404.

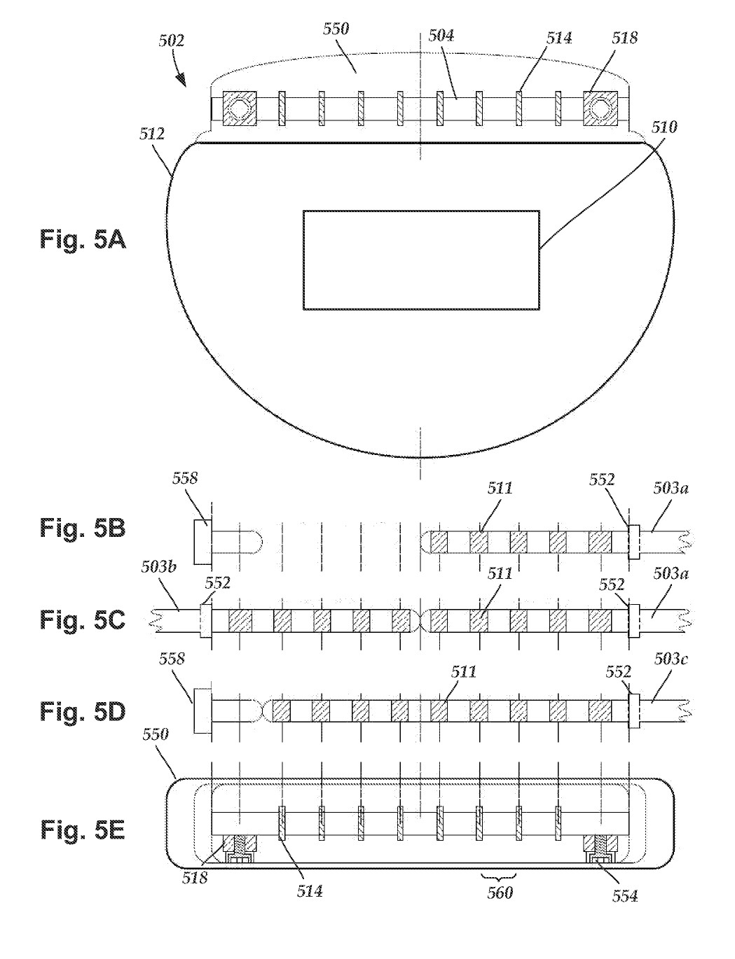

FIG. 5A is a schematic side view of another embodiment of a control module 502. The control module 502 includes a sealed housing 512, an electronic subassembly 510, and a header arrangement 550 similar in structure and function to the sealed housing 412, the electronic subassembly 410 and the header arrangement 450 respectively, of the control module 402 as shown in FIG. 4A. The header arrangement 550 also includes a retention assembly 518 to secure a lead (e.g. lead 503a shown in FIG. 5B) within the header arrangement 550. The retention assembly 518 may be structurally and functionally similar to the retention assembly 418.

The header arrangement 550 also includes a receiving lumen 504 and one or more contacts 514 disposed around the circumference of the receiving lumen 504. The receiving lumen 504 is similar to the receiving lumen 404 of the control module 402. Further, the receiving lumen 504 includes at least two openings, i.e., a first opening and a second opening. As shown, the contacts 514 are disposed within the receiving lumen 504, such that each contact 514 is at a uniform distance from the adjacent contacts. The distance between each pair of contacts 514 is the same.

FIG. 5B is a schematic side view of one embodiment of an arrangement of the lead 503a and a plug (a “short plug”) 558 for insertion in the receiving lumen 504 of the control module 502. The lead 503a includes a number of lead terminals 511 disposed at a proximal end portion of the lead 403a. Further, the lead 403a includes at least four terminals 511. The lead 503a can include any number of terminals 511, depending on number of contacts 511 or the size and structure of the receiving lumen 504. Further, a flange 552 similar to the flange 452 is disposed on the proximal end portion of the lead 503a which prevents insertion of the lead 503a too deep within the receiving lumen.

When a single lead 503a is inserted into a first opening of the receiving lumen 504, then the short plug 558 may be inserted into the second opening, thereby blocking the second opening. The short plug 558 is functionally similar to the long plug 456 shown in FIG. 4B, but may differ in size from the long plug 456. In the illustrated embodiment, the short plug 558 does not extend past any of the contacts 514 in the receiving lumen 504. The short plug 558 may only extend past the retention assembly 518. In alternative embodiments, the short plug 558 can extend past one, two, three, or more contacts 514. The short plug 558 may be formed using the same biocompatible material as that of the long plug 456.

FIG. 5C is a schematic side view of one embodiment of an arrangement of two leads (i.e., leads 503a-503b) for insertion in the receiving lumen 504 of the control module 502. In this embodiment, both the leads 503a and 503b are short leads and can be inserted into the receiving lumen 504 from the first and the second openings, respectively. Each of the leads 503a, 503b can include a flange 552 and one or more lead terminals 511 disposed along a proximal end portion of the lead. The lead terminals 511 of both the leads 503a, 503b are inserted so that they are aligned with the respective contacts 514 when disposed within the receiving lumen 504. The size of the tip portion of each of the leads 503a, 503b may be half of the distance between a pair of the contacts 514.

FIG. 5D is a schematic side view of one embodiment of an arrangement of a long lead 503c and a short plug 558 for insertion in the receiving lumen 504 of the control module 502. The long lead 503c includes one or more terminals 511 which are more than half the number of the contacts 514 within the receiving lumen 504. For example, the number of terminals 511 can be five, six, eight, and so forth. The long lead 503c is disposed in the receiving lumen 504 such that the long lead 503c can extend past more than half of the contacts 514 disposed within the receiving lumen 504. Hence, only one long lead 503c can be disposed in the receiving lumen 514. The short plug 558 can be inserted into the second opening within the receiving lumen 504 when the long lead 503c is inserted into the first opening.

FIG. 5E is a schematic partial-cross-sectional top view of the header arrangement 550 of the control module 502. The header arrangement 550 includes the contacts 514, which are uniformly placed such that the adjacent contacts 514 in a pair are separated by a first distance 560. The first distance 560 may be greater or smaller than a width of the contact 514. The header arrangement 550 also includes the retention assembly 518 similar to the retention assembly 418 of the control module 402. Further, a fastener 554 may be disposed into the retention assembly 418. The fastener 554 can be tightened against the lead to securely hold the lead within the header arrangement 550.

The structure of the above embodiments enables two leads, such as the leads 503a, 503b, to be simultaneously inserted into a single receiving lumen 504 with two ports or openings. In situations where only one lead is inserted, the other opening may receive a short plug 558 or long plug 456. The use of a plug can be advantageous for several reasons, such as by reducing or preventing unintended access to the terminals 511 and the contacts 514 within the receiving lumen 504 from open end, reducing or preventing flow of contaminants into the receiving lumen, and the like.

At least some embodiments employ include a device or method for facilitating insertion of the leads within the receiving lumen 504 to the proper depth so that the terminals 511 of one or more of the leads 503a-503c operationally communicate with the contacts 514 of the receiving lumen 504. For example, a visual marker may be used to communicate an acceptable or proper insertion depth. Other structures, such as flange 552, may be used to prevent over insertion into the receiving lumen 504. When the lead is inserted into the receiving lumen 404, the flange 552 may ensure that the terminals 511 and the contacts 514 are aligned with respect to each other. Other embodiments use the plug such as the long plug 456 or the short plug 558 to perform this operation, such as a plug inserted from one end of the receiving lumen 504 obstructing a lead inserted from the other end of the receiving lumen 504 proceeding beyond a certain depth of the receiving lumen 504.

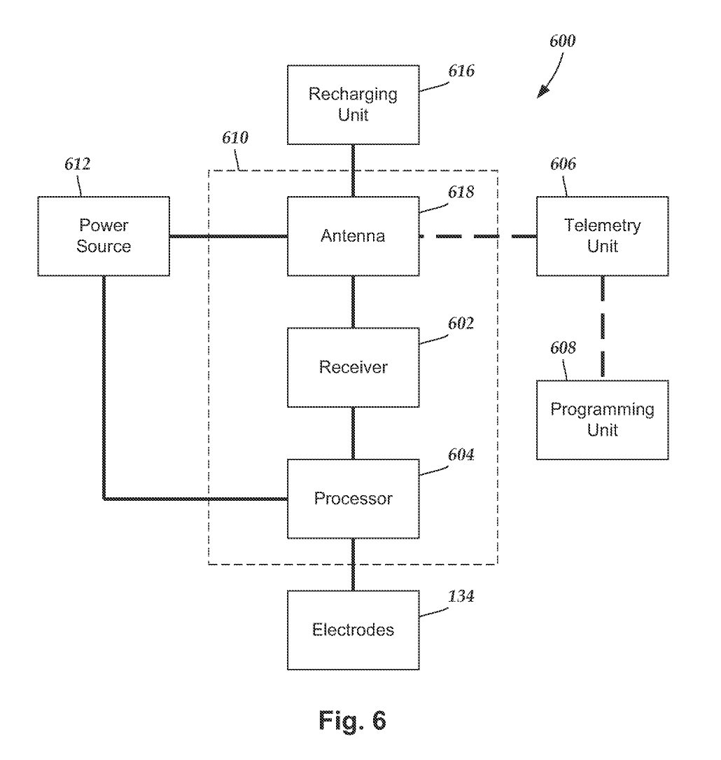

FIG. 6 is a schematic overview of one embodiment of components of an electrical stimulation system 600 including an electronic subassembly 610 disposed within a control module. It will be understood that the electrical stimulation system can include more, fewer, or different components and can have a variety of different configurations including those configurations disclosed in the stimulator references cited herein.

Some of the components (for example, a power source 612, an antenna 618, a receiver 602, and a processor 604) of the electrical stimulation system can be positioned on one or more circuit boards or similar carriers within a sealed housing of an implantable pulse generator, if desired. Any power source 612 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference.

As another alternative, power can be supplied by an external power source through inductive coupling via the optional antenna 618 or a secondary antenna. The external power source can be in a device that is mounted on the skin of the user or in a unit that is provided near the user on a permanent or periodic basis.

If the power source 612 is a rechargeable battery, the battery may be recharged using the optional antenna 618, if desired. Power can be provided to the battery for recharging by inductively coupling the battery through the antenna to a recharging unit 616 external to the user. Examples of such arrangements can be found in the references identified above.

In one embodiment, electrical current is emitted by the electrodes 134 on the paddle or lead body to stimulate nerve fibers, muscle fibers, or other body tissues near the electrical stimulation system. The processor 604 is generally included to control the timing and electrical characteristics of the electrical stimulation system. For example, the processor 604 can, if desired, control one or more of the timing, frequency, strength, duration, and waveform of the pulses. In addition, the processor 604 can select which electrodes can be used to provide stimulation, if desired. In some embodiments, the processor 604 selects which electrode(s) are cathodes and which electrode(s) are anodes. In some embodiments, the processor 604 is used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 608 that, for example, allows modification of pulse characteristics. In the illustrated embodiment, the processor 604 is coupled to a receiver 602 which, in turn, is coupled to the optional antenna 618. This allows the processor 604 to receive instructions from an external source to, for example, direct the pulse characteristics and the selection of electrodes, if desired.

In one embodiment, the antenna 618 is capable of receiving signals (e.g., RF signals) from an external telemetry unit 606 which is programmed by the programming unit 608. The programming unit 608 can be external to, or part of, the telemetry unit 606. The telemetry unit 606 can be a device that is worn on the skin of the user or can be carried by the user and can have a form similar to a pager, cellular phone, or remote control, if desired. As another alternative, the telemetry unit 606 may not be worn or carried by the user but may only be available at a home station or at a clinician's office. The programming unit 608 can be any unit that can provide information to the telemetry unit 606 for transmission to the electrical stimulation system 600. The programming unit 608 can be part of the telemetry unit 606 or can provide signals or information to the telemetry unit 606 via a wireless or wired connection. One example of a suitable programming unit is a computer operated by the user or clinician to send signals to the telemetry unit 606.

The signals sent to the processor 604 via the antenna 618 and the receiver 602 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the electrical stimulation system 600 to cease operation, to start operation, to start charging the battery, or to stop charging the battery. In other embodiments, the stimulation system does not include the antenna 618 or receiver 602 and the processor 604 operates as programmed.

Optionally, the electrical stimulation system 600 may include a transmitter (not shown) coupled to the processor 604 and the antenna 618 for transmitting signals back to the telemetry unit 606 or another unit capable of receiving the signals. For example, the electrical stimulation system 600 may transmit signals indicating whether the electrical stimulation system 600 is operating properly or not or indicating when the battery needs to be charged or the level of charge remaining in the battery. The processor 604 may also be capable of transmitting information about the pulse characteristics so that a user or clinician can determine or verify the characteristics.

The above specification provides a description of the manufacture and use of the invention. Since many embodiments of the invention can be made without departing from the spirit and scope of the invention, the invention also resides in the claims hereinafter appended.

What is claimed as new and desired to be protected by Letters Patent of the United States is:

1. An implantable control module for coupling to one or more implantable stimulation leads, the control module comprising:
a sealed housing;
an electronic subassembly disposed in the housing;
a header arrangement coupled to the housing, the header arrangement comprising at least one receiving lumen and a plurality of contacts disposed within the at least one receiving lumen, wherein each receiving lumen has two opposing openings which are each configured and arranged for receiving an implantable stimulation lead through the opening and within the receiving lumen, wherein the plurality of contacts are arranged within the at least one receiving lumen to make contact with terminals on the implantable stimulation lead received in the receiving lumen; and
a plurality of feedthrough elements extending from the header arrangement into the sealed housing and electrically coupling the plurality of contacts of the header arrangement with the electronic subassembly.

2. The control module of claim 1, wherein the at least one receiving lumen comprises a first receiving lumen and the plurality of contacts comprises a plurality of first contacts, wherein the first contacts are arranged within the first receiving lumen.

3. The control module of claim 2, wherein the first contacts are uniformly spaced apart.

4. The control module of claim 2, wherein each adjacent pair of the first contacts are spaced apart by a first distance except a middle pair of the first contacts which are spaced apart by second distance that is different from the first distance.

5. The control module of claim 4, wherein the second distance is greater than the first distance.

6. The control module of claim 4, wherein the second distance is double the first distance.

7. The control module of claim 1, further comprising a plug insertable into any one of the openings of the at least one receiving lumen of the header arrangement.

8. The control module of claim 7, wherein the plug is configured and arranged so that, when fully inserted into one of the openings, the plug extends past at least half of the contacts within the receiving lumen in which the plug is inserted.

9. The control module of claim 7, wherein the plug is configured and arranged so that, when fully inserted into one of the openings, the plug does not extend past any of the contacts within the receiving lumen in which the plug is inserted.

10. A kit, comprising:
the control module of claim 1; and
at least one lead comprising a proximal end insertable into one of the at least one receiving lumen of the control module.

11. The kit of claim 10, wherein the at least one lead further comprises a flange disposed along the proximal end of the lead and configured and arranged to halt further insertion of the lead into the at least one receiving lumen of the control module when the flange makes contact with the header arrangement of the control module.

12. The kit of claim 10, wherein the at least one lead comprises a first lead and a second lead, wherein the first and second leads are configured and arranged to be received together within a same receiving lumen by insertion of the first and second leads through respective ones of the two openings of the receiving lumen.

13. The kit of claim 10, further comprising a plug insertable into any one of the openings of the at least one receiving lumen of the header arrangement.

14. The kit of claim 13, wherein the plug is configured and arranged so that, when fully inserted into one of the openings, the plug extends past at least half of the contacts within the receiving lumen in which the plug is inserted.

15. The kit of claim 13, wherein the plug is configured and arranged so that, when fully inserted into one of the openings, the plug does not extend past any of the contacts within the receiving lumen in which the plug is inserted.

16. A method of implanting an electrical stimulation lead, the method comprising:
providing the control module of claim 1;
inserting a proximal end portion of a first electrical stimulation lead into a first opening of a first receiving lumen of the at least one receiving lumen of the control module; and
implanting the first electrical stimulation lead and the control module.

17. The method of claim 16, further comprising inserting a proximal end portion of a second electrical stimulation lead into a second opening of the first receiving lumen of the control module.

18. The method of claim 16, further comprising inserting a plug into a second opening of the first receiving lumen of the control module.

19. The method of claim 18, wherein the plug is configured and arranged so that, when fully inserted into one of the openings, the plug extends past at least half of the contacts within the receiving lumen in which the plug is inserted.

20. The method of claim 18, wherein the plug is configured and arranged so that, when fully inserted into one of the openings, the plug does not extend past any of the contacts within the receiving lumen in which the plug is inserted.
